(19) United States
(12) Patent Application Publication
(10) Pub. No.: US 2025/0354897 A1
(43) Pub. Date: Nov. 20, 2025

(54) AUTOMATED FLOORBOARD DOOR FOR ACQUIRING ENVIRONMENTAL SAMPLES AND DATA FROM WITHIN AN IMPLEMENT

(71) Applicant: Precision Land Service, LLC, Adel, IA (US)

(72) Inventor: Scott Ryan Mattes, Adel, IA (US)

(73) Assignee: Precision Land Service, LLC, Adel, IA (US)

(21) Appl. No.: 18/668,782

(22) Filed: May 20, 2024

(51) Int. Cl. G01N 1/08; E05F 1/10; E05F 15/00; G01N 33/24

(52) U.S. Cl. CPC G01N 1/08; E05F 1/10; E05F 15/00; G01N 33/245; E05Y 2900/531

(57) ABSTRACT

An automated floorboard door for acquiring test data from an environment outside of an implement using a testing device and tester within the implement is disclosed. The automated floorboard door actuates from a closed position to an open position for the testing device to acquire data from the testing area below through an opening in the floorboard. Upon completion of testing, the automated floorboard door closes and seals the opening in the floorboard thereby closing the cab to the environment outside the implement. An enclosure on the implement protects a motor operating the testing device from dust, debris and presenting a fire hazard in the testing area.

### FIELD OF THE INVENTION

The present invention relates to acquiring testing data from within an implement. More particularly, but not exclusively, the present invention relates to systems and methods for acquiring environmental data from within an implement through an automated floorboard door.

### BACKGROUND

Testing devices are often used for quickly acquiring data from an environment. Vehicle or implement mounted testing devices allow for increased sampling efficiencies. Placement of the testing device or equipment within the cab of an implement provides further sampling and testing efficiencies, while providing the implement operator and the equipment with all the benefits of a quiet, dust-free, and climate controlled operating environment. A hole or port in the floorboard of the implement allows for taking samples from the ambient environment using equipment installed within the cab of the implement. However, an open hole in the floorboard of an implement is neither safe or effective at maintaining the benefits of a closed cab environment. Therefore, there is a need to provide an automated floorboard door that is safe, maintains the closed cab environment, and allows testing devices to acquire data from the ambient environment and from within the cab of the implement.

In at least one application, testing devices may be used for obtaining ambient environmental data and soil samples for analyzing soil conditions. In one aspect, testing devices may be used to acquire soil samples from within an agricultural field. For example, testing devices, such as a soil probe, have been mounted to vehicles to make data collection easier over difficult terrain. In some instances, testing devices are mounted to an all-terrain vehicle (ATV) or utility task vehicle (UTV) to improve the speed and efficiency of obtaining soil data. Testing device mounting configurations range from being externally mounted such as outside an operator's cab or internally mounted such as inside an operator's cab. Testing devices mounted externally are dusty, loud, difficult to access and lead to unwanted operator fatigue. This has led to testing devices being mounted inside the cab of a vehicle, such as the cab of an ATV/UTV, to minimize dust, reduce noise, ease access to and operation of, and improve operator comfort while lessening operator fatigue. This is often accomplished by mounting the testing device on the passenger side of the implement and creating a hole or port in the passenger floorboard for the testing device to acquire data from outside of the implement. If the port is always open to the exterior, dust, debris, insects, moisture, and noise can enter the cab. In the case of acquiring soil samples, crop residue and debris, such as corn stalks, may become lodged in and enter through the port, which can impact operation of and result in damage to the testing device thereby increasing operational downtime, repairs, and costs, and decreasing operational efficiency. Therefore, a need exists in the art to seal or close off the port in the floorboard of the implement between data samplings to limit dust, debris, insects, moisture, and noise from entering the cab, improve the operator's working environment, and decrease operator fatigue.

In some applications, testing devices are powered by the implement. This may include electrical and/or hydraulic connections to electrical and hydraulic outputs for the implement. In other applications, a gas-powered engine, separate from the implement, may be used for powering a hydraulic pump for hydraulically operating mechanized members of the testing device or for providing electricity to the testing device for collecting test data. Externally mounted gas engines present a fire hazard as dry tinder, such as debris, contacts hot portions of the motor and exhaust. Also, gas engines and hydraulic pumps operating in dusty, dirty, and debris-ridden environments need to be serviced often to maintain their operational readiness, reliability, and life expectancy. A gas-powered engine can be enclosed to address these issues; however, maintaining and servicing engines and hydraulic pumps in enclosed environments is difficult and often leads to less as opposed to more care given to the engine and hydraulic pump. Therefore, a need exists in the art to provide an enclosure for a gas-powered engine and hydraulic pump to protect them from dusty, dirty, and debris-ridden environments while reducing the fire risk. Additionally, a need exists in the art to provide an enclosure for an engine and hydraulic pump that allows for easy access to service and maintain the engine and hydraulic pump.

### SUMMARY

Therefore, it is a primary object, feature, or advantage of the present invention to improve over the state of the art.

It is a further object, feature, or advantage of the present invention to provide an automated floorboard door in the cab of an implement.

It is yet a further object, feature, or advantage of the present invention to provide an automated floorboard door in the cab of an implement for collecting data with a testing device from within the cab and from the environment outside the cab.

It is a still further object, feature, or advantage of the present invention to provide an automated floorboard door for a tester to acquire data where the door closes and seals between data and/or material samplings to limit dust, noise, insects, moisture, and debris from the outside environment from entering the cab of the implement and fouling operation of the probe and/or impairing or fatiguing the implement operator.

It is at least a further object, feature, or advantage of the present invention to provide an automated floorboard door for a tester where the door closes and seals between data samplings to limit dust, noise, insects, moisture, and debris from the outside environment from entering the cab of the implement and fouling operation of the tester and/or impairing or fatiguing the implement operator.

It is yet a further object, feature, or advantage of the present invention to provide an automated floorboard door for a testing device where the door closes and seals between testing to limit dust, noise, insects, moisture, and debris from the outside environment from entering the cab of the implement and fouling operation of the testing device and/or impairing or fatiguing the implement operator.

Another object, feature, or advantage is to provide a control stand for mounting and height adjustment of a testing device mounted to the floorboard of an implement.

Yet another object, feature, or advantage is to provide an enclosure for an engine and hydraulic pump operating a testing device that allows for easy access to service and maintain the engine and hydraulic pump.

In at least one exemplary aspect, automated floorboard door for acquiring test data from an environment outside of an implement using a testing device within the implement is disclosed. The automated floorboard door includes at least one floorboard door disposed in an opening in a floorboard of an implement. The at least one floorboard door has a closed position occupying the opening in the floorboard of the implement and an open position with the least one floorboard door removed from the opening in the floorboard. The at least one floorboard door is actuated to the open position for acquiring data with the testing device through the opening.

In at least one embodiment of another exemplary aspect of the present invention, a soil probe implement is disclosed. The soil probe implement includes a motorized implement having a cab with operator controls and a soil probe operably mounted within the cab. An opening is in a floorboard of the implement for the soil probe to pass through in probing contact with the soil. The at least one floorboard is door disposed in the opening in the floorboard of the implement. The at least one floorboard door is actuated to an open position for the soil probe to pass through to the soil below and a closed position for closing the opening in the floorboard when the soil probe is retracted back into the cab.

In another exemplary aspect, an automated method for acquiring test data from the environment outside of an implement using a testing device within the implement is disclosed. The method includes such steps as providing at least one floorboard door disposed in an opening in a floorboard of the implement, where the at least one floorboard door has a closed position occupying the opening in the floorboard of the implement and an open position with the least one floorboard door removed from the opening in the floorboard. Other exemplary steps include actuating the at least one floorboard door to the open position for movement of the testing device through the opening, acquiring data from outside the implement with the testing device, retracting the testing device into the implement, and actuating the at least one floorboard door to the closed position.

One or more of these and/or other objects, features, or advantages of the present invention will become apparent from the specification and claims that follow. No single embodiment need provide each and every object, feature, or advantage. Different embodiments may have different objects, features, or advantages. Therefore, the present invention is not to be limited to or by any objects, features, or advantages stated herein.

### BRIEF DESCRIPTION OF THE DRAWINGS

Illustrated embodiments of the disclosure are described in detail below with reference to the attached drawing figures, which are incorporated by reference herein.

FIG. 1 is a perspective view of an implement in accordance with an exemplary aspect of the present invention.

FIGS. 2A-B are perspective views of a testing device in accordance with an exemplary aspect of the present invention.

FIGS. 3A-D are top perspective views of an automated floorboard door and testing device in accordance with an exemplary aspect of the present invention.

FIGS. 4A-C are bottom perspective views of the automated floorboard door and testing device in accordance with exemplary aspects of the present invention.

FIGS. 5A-C are perspective views of a biasing system for the automated floorboard door in accordance with an exemplary aspect of the present invention.

FIGS. 6A-D are perspective views of a testing device motor enclosure in accordance with exemplary aspects of the present invention.

FIG. 7 is a flowchart illustrating an exemplary method of the present invention.

### DETAILED DESCRIPTION

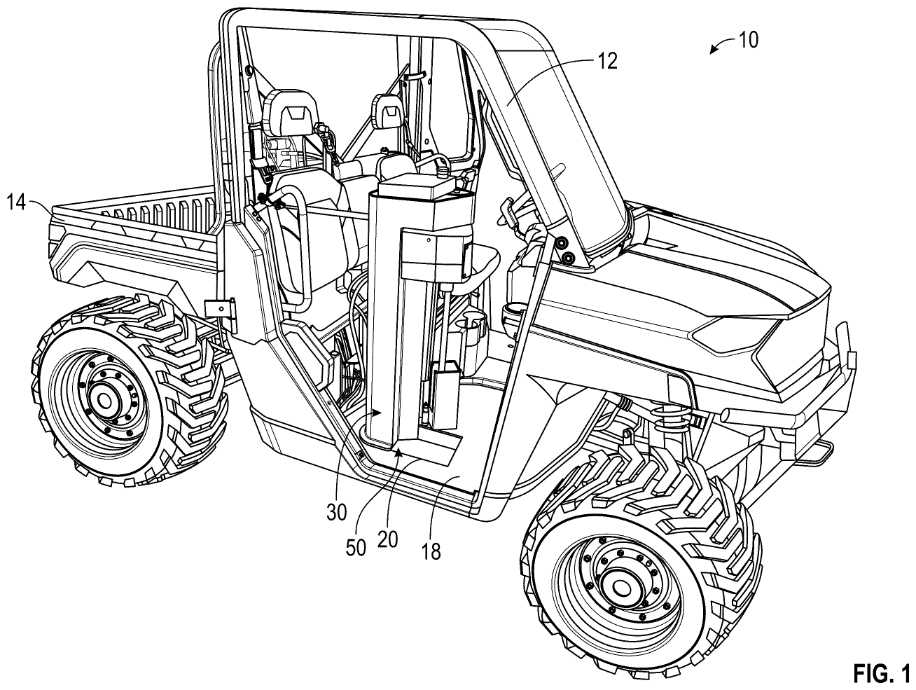

FIGS. 1-7 provide various exemplary aspects pertaining to the present invention. FIG. 1 illustrates one example of an implement 10 in accordance with an exemplary aspect of the present invention. Implement 10 may be any implement having a cab 12. In at least one example, implement 10 may be any implement having a cab 12, such as an all-terrain vehicle (ATV), utility task vehicle (UTV), or other vehicle types having a cab 12. In at least one other aspect, implement 10 may be any vehicle having a cab 12 and a bed 14. Implement 10 may be a vehicle having a cab 12, wherein implement 10 is configured for towing a trailer (not shown) in addition to or replacing bed 14. Operator controls 16 are configured within cab 12 for controlling operation of implement 10. Operator controls 16 may be located on the driver side of implement 10. Cab 12 may also include a driver and passenger side and a floorboard 18 on each side. An opening 20 in floorboard 18 may be provided for acquiring testing data from the environment outside of the implement 10 with a testing device located within cab 12 of implement 10. Opening 20 may be configured in floorboard 18 on the passenger side of implement 10.

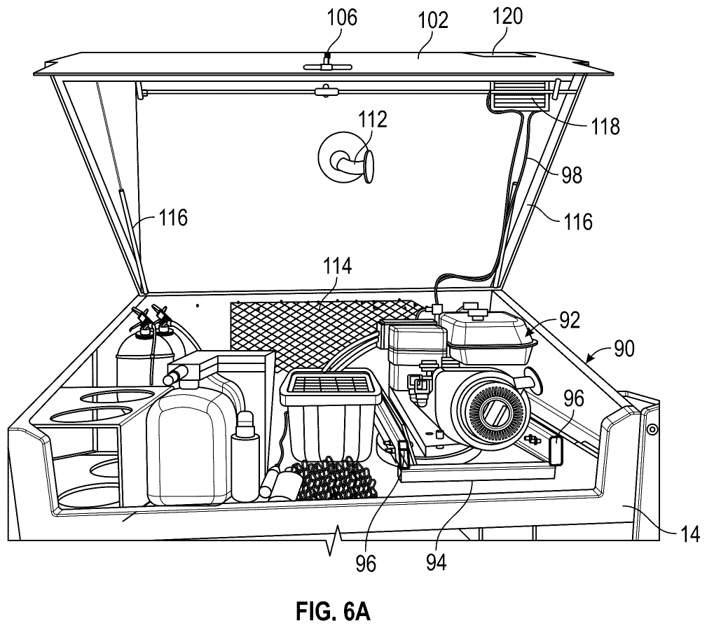

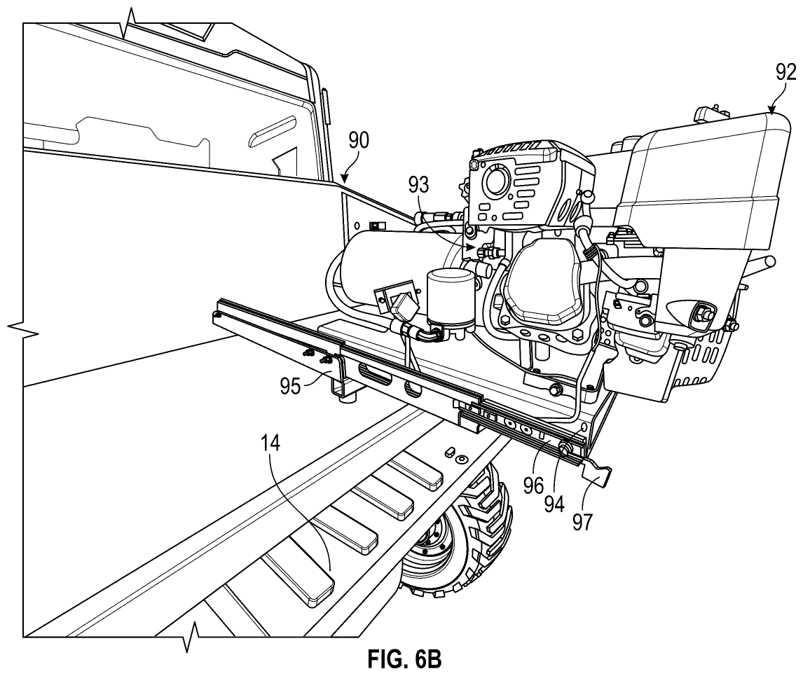

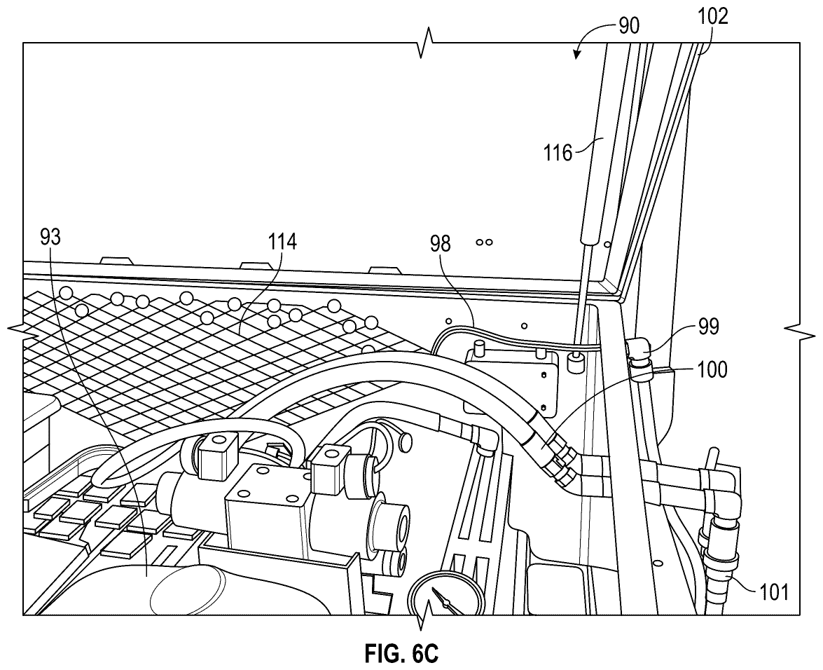

In accordance with one aspect of the invention, testing data is acquired through opening 20 with a testing device 30. For example, testing device 30, shown by way of example, in FIGS. 1, 2A-2B, 3B-3D, and 5A-5C, may be mounted within cab 12. In various examples, implement 10 may be configured as a testing implement, such as a soil probe implement. For example, testing device 30 may include a tester 34, such as a probe, for acquiring a sample through opening 20 or include tester 34, such as a sensor, for acquiring data through opening 20. One probe type for tester 34 may be a soil probe for acquiring a soil sample, such as the soil sampler sold by Wintex Agro USA and shown at www.wintexagrousa.com, which all features, functions, and operations of the soil probe are incorporated by reference in their entirety herein. Testing device 30 and tester 34 from other manufacturers are also contemplated. For example, tester 34 may be a soil pH sensor, probe for collecting media (soil, vegetation, concrete, aggregate, etc.) samples, a moisture sensor, a conductivity sensor, a temperature sensor, a nutrients sensor, or a salinity sensor. Tester 34 may be configured with sonar, radar, laser, spectrometer, or other hardware for acquiring samples and/or data regarding the testing area beneath implement 10. In at least one example, testing device 30 is mounted to floorboard 18 and proximate opening 20 on the passenger side of cab 12. Testing device 30 may be secured within and to cab 12 with one or more testing device stands 44 and one or more mounting plates 48. Testing device 30 may include a carriage 32 that carries tester 34. In one aspect, carriage 32 is operatively attached to a tower rail 46 that is hydraulically driven up and down by testing device 30. Carriage 32 may be configured to rotate and/or plunge tester 34, akin to a drill, during acquisition of a sample or data. Testing device 30 includes electrical connections 40 and hydraulic connections 42 for operating testing device 30, tower rail 46, and carriage 32. A motor 92 powering a hydraulic pump 93, shown in FIGS. 6A-6C, includes electrical connections 98 and hydraulic connections 100 operably connected to electrical connections 40 and hydraulic connections 42 of testing device 30. Electrical connections 98 of motor 92 and testing device 30 may be operably connected to operator controls 16 in cab 12 of implement 10. In another aspect, electrical connections 40 and hydraulic connections 42 may be operably connected to the electrical and hydraulic system of implement 10. Tower rail 46 and carriage 32 move vertically down (toward the ground) in a first direction and vertically up (away from ground) in a second direction opposite the first direction to insert tester 34 into the ground and retrieve tester 34 and a sample from the testing area below, such as a soil or other testing area sample. An ejector 36 ejects the sample into a sample box 38 for collecting and subsequent testing. In addition to the hydraulic range of motion of tower rail 46, mounting plates 48 may be positioned between the base of testing device 30 and floorboard 18 to control the distance of separation and thereby the plunge depth of tester 34 relative to opening 20 in floorboard 18 and the ground below. Increasing the number of mounting plates 48 increases the distance between the carriage 32 and floorboard 18, thereby decreasing the plunge depth of tester 34 relative to opening 20 in floorboard 18 and the ground below. Similarly, decreasing the number of mounting plates 48 decreases the distance between the carriage 32 and floorboard 18, thereby increasing the plunge depth of tester 34 relative to opening 20 in floorboard 18 and the ground below. In one application, tester 34 may be moved from within cab 12 to a position closer to the ground for acquiring data from the testing area and then retrieved back into cab 12 through opening 20 in floorboard 18. In another application, tester 34 may maintain its position within cab 12 and acquire data from the testing area below through opening 20 in floorboard 18.

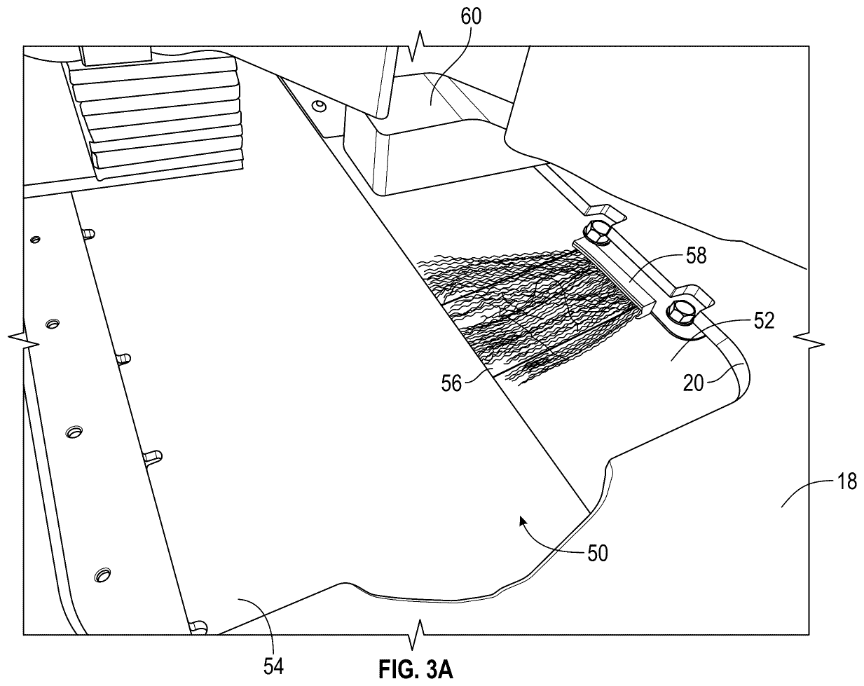

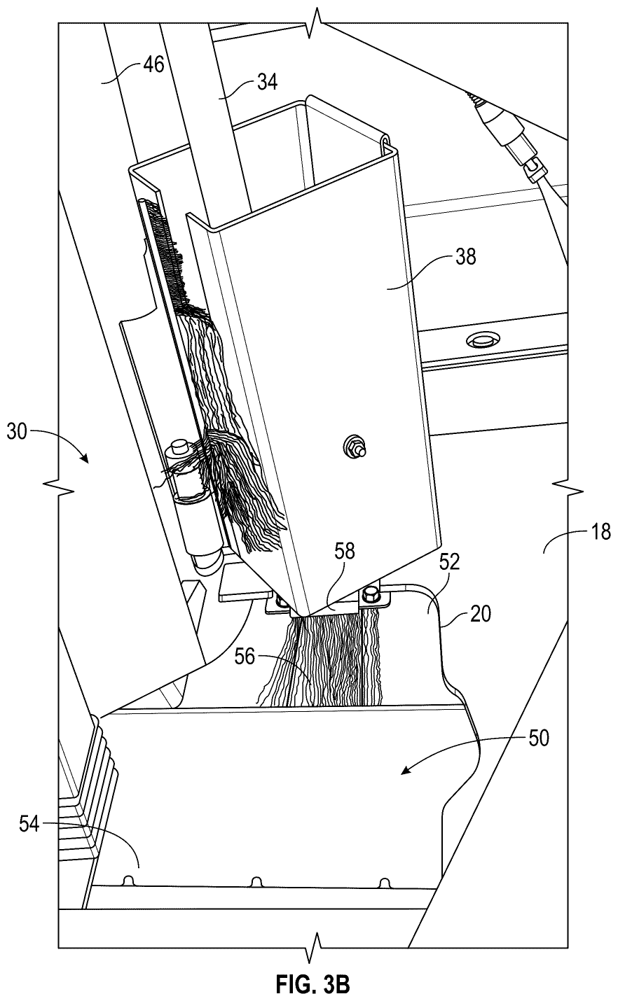

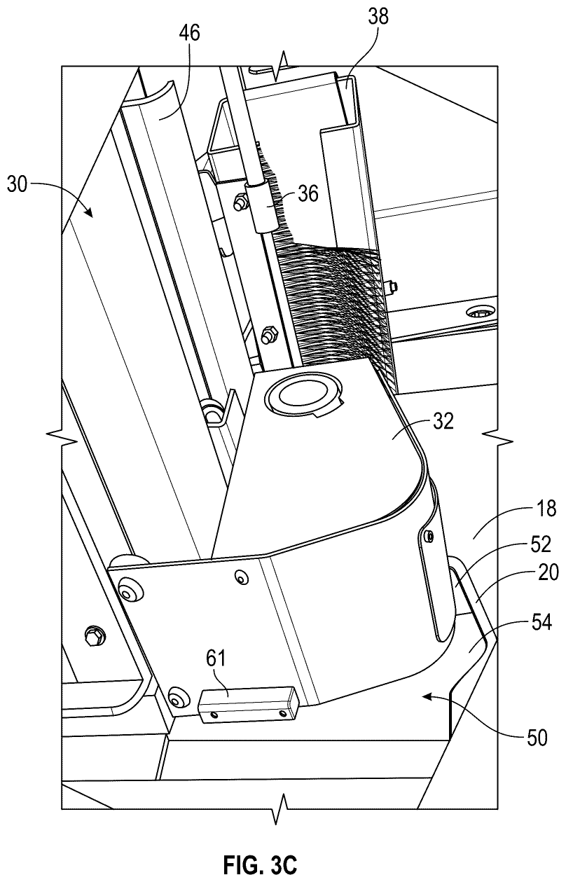

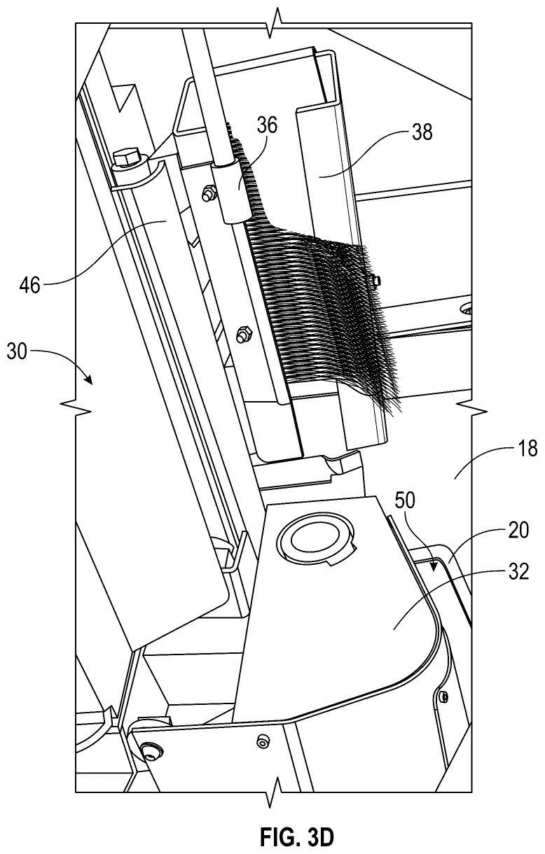

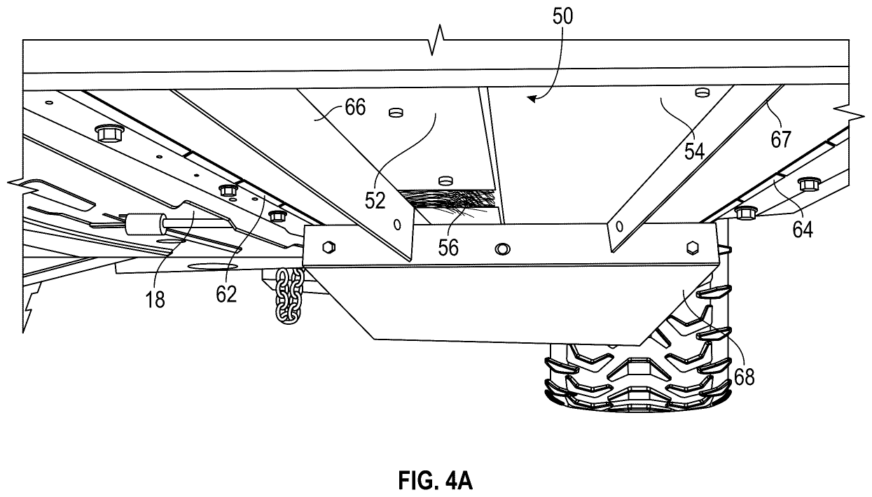

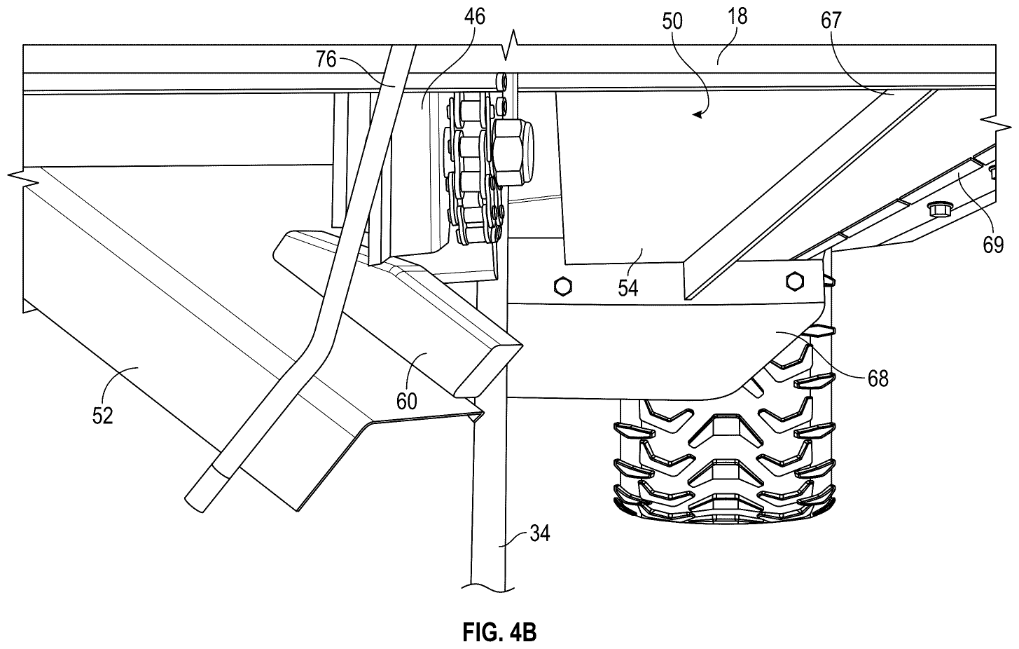

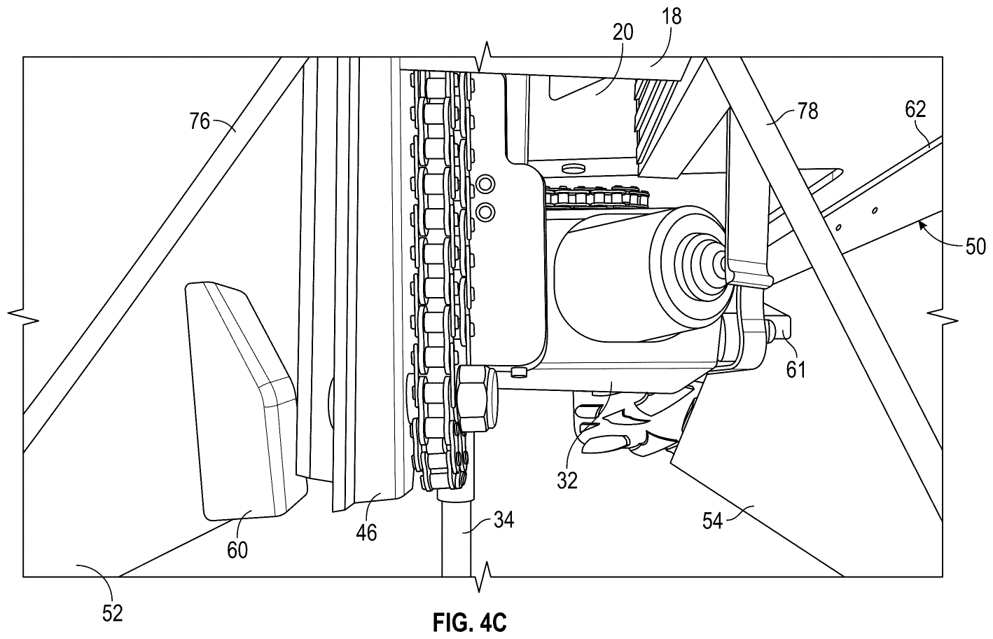

Disposed within opening 20 in floorboard 18 is an automated floorboard door 50, shown by way of example in FIGS. 1, 2A, 3A-3D, and 4A-4C. In one aspect, floorboard door 50 includes a pair of doors 52, 54, but may be configured having a single door or multiple doors. Door 52 is rotatably attached adjacent opening 20 to floorboard 18 via hinge 62. Door 52 may be configured to slide in a plane parallel to floorboard 18 for moving into and out of a covering position relative to opening 20. Similarly, door 54 is rotatably attached adjacent opening 20 to floorboard 18 via hinge 64. Door 54 may be configured to slide in a plane parallel to floorboard 18 for moving into and out of covering relation relative to opening 20. Doors 52, 54 are configured, in at least one application of the present invention, to rotate away from opening 20 in floorboard 18, either by rotating upward or downward. In one aspect, doors 52, 54 rotate downward away from cab 12 as shown in FIGS. 4B-4C. Doors 52, 54 may alternatively be configured to rotate upward into the interior of cab 12. Doors 52, 54 occupy opening 20 in floorboard 18 in their closed position and do not occupy opening 20 in their opening position. In one aspect, doors 52, 54 are generally parallel with floorboard 18 in their closed position shown, for example, in FIG. 4A and generally orthogonal to floorboard 18 in their open position shown, for example, in FIG. 4C. Doors 52, 54 are generally coplanar in the closed position as shown, for example, in FIGS. 3A and 4A. Doors 52, 54 are noncoplanar in the open position as shown, for example, in FIGS. 3D and 4C. Doors 52, 54 move from the generally coplanar position when closed to the noncoplanar position when opened. The opening angle of doors 52, 54 may be adjusted as needed. For example, doors 52, 54 may be configured to have an opening angle relative to their closed position ranging from 70-110 degrees. A port 56, such as an opening sufficiently sized as a port to receive tester 34, may be configured having a door of the automated floorboard door 50. Port 56 may also be sufficiently sized as a tester port to receive a tester, such as tester 34. In one aspect, port 56 is configured in door 52. A brush 58 may be disposed in port 56. Brush 58 is replaceable when it wears out or bristles no longer function correctly. In one aspect, brush 58 is attached to door 52 proximate port 56 to place brushes of brush 58 in covering relation to port 56 and an interior edge of opposing door 54. A cam member 60 is attached to the automated floorboard door 50 for being acted upon to actuate automated floorboard door 50 from the closed to open position. In one aspect, door 52 includes cam member 60 shown in FIGS. 3A and 4B-4C. Cam member 60 may be configured with at least one profiled surface for increasing the opening rate and opening angle of door 52 when actuated from the closed to open position. Door 54 may also include a cam member, similar to cam member 60, for controlling the rate of opening and opening angle of door 54 when actuated. FIG. 3C shows a cam member 61 attached to carriage 32. Cam member 61 acts on door 54 and actuates door 54 to the open position (FIGS. 3D and 4C) when tower rail 46 and carriage 32 carrying tester 34 descends through opening 20 for acquiring a sample or data from the testing area below. One or more deflectors shown, for example in FIGS. 4A-4B, may be configured about opening 20 on the underside of floorboard 18 and on the underside of doors 52, 54 for deflecting debris, such as objects or debris within the testing area below opening 20 in floorboard 18 of implement 10. Deflectors 66, 67 also act as a stiffening member to add rigidity to doors 52, 54. In one aspect, the underside of door 52 includes a deflector 66 extending downward generally orthogonal to door 52 and parallel to hinge 62. The length of deflector 66 may be configured to extend from opposing edges of door 52. Similarly, the underside of door 54 includes a deflector 67 extending downward generally orthogonal to door 54 and parallel to hinge 64. The length of deflector 67 may be configured to extend from opposing edges of door 54. When doors 52, 54 are in a closed position as shown, for example, in FIG. 4A, deflectors 66, 67 are disposed in a generally vertical position. When doors 52, 54 are in an open position as shown, for example, in FIG. 4C, deflectors 66, 67 are disposed in a generally horizontal position. In another aspect, one or more deflectors may be attached on the underside of floorboard 18 at the leading edge or trailing edge of opening 20. In one aspect, a deflector 68 may be attached to the underside of floorboard 18, adjacent and parallel to the leading edge of opening 20. Deflector 68 may be angled forward away from opening 20 at an angle to deflect objects and debris within the testing area below away from opening 20.

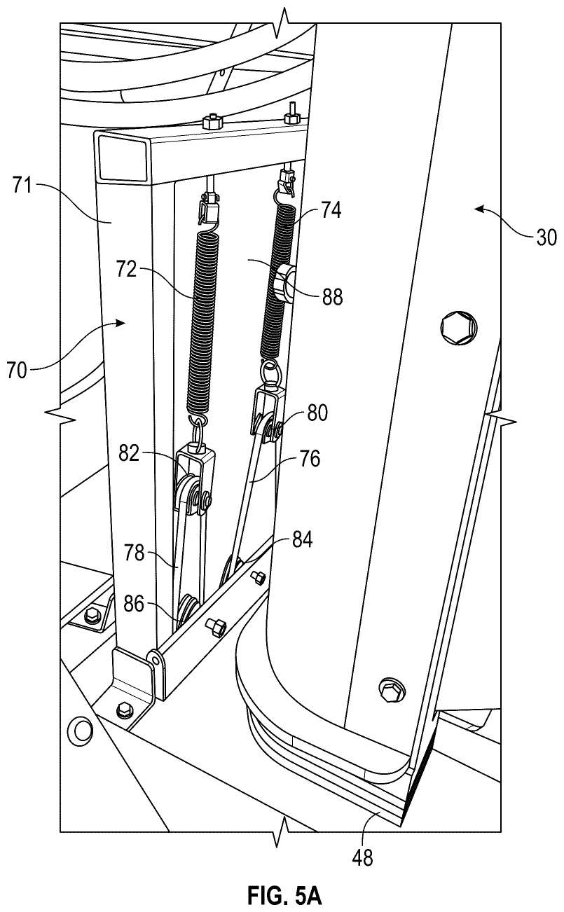

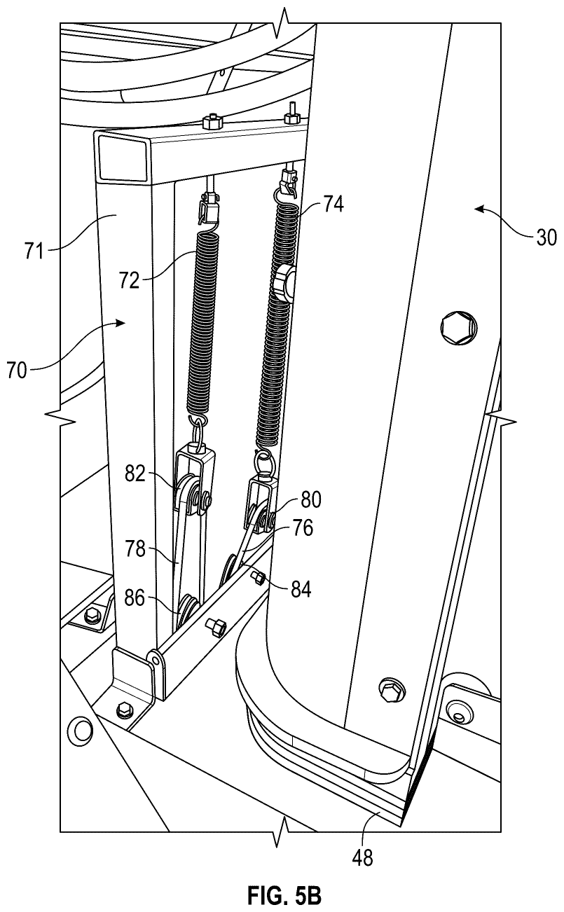

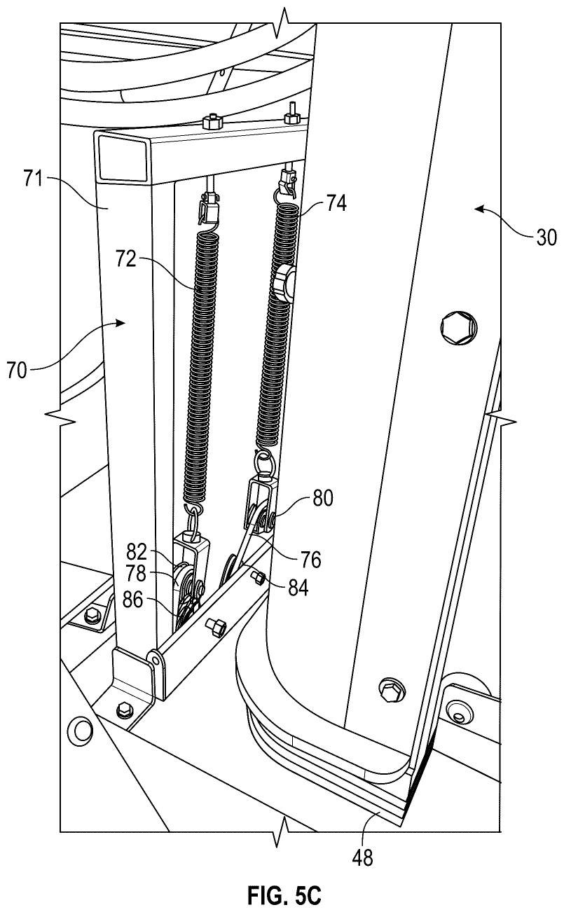

FIGS. 5A-5C illustrate a door biasing system 70 for biasing doors 52, 54 to the closed position shown, for example, in FIGS. 3A and 4A. In one aspect, each door has a separate biasing system for biasing each door to the closed position shown in FIGS. 3A, 3B, and 4A. In another aspect, both doors are controlled by a single biasing system for biasing both doors to the closed position. Door biasing system 70 includes a housing 71 for enclosing one or more components of door biasing system 70. Housing 71 may include one or more frame members and one or more enclosing panels 88. In one aspect, a front panel (not shown for illustrating door biasing system 70) and a rear panel 88 may be removably and magnetically attached to housing 71 using one or more magnets (not shown), latches, screens, pins or other securements. A first biasing system for door 52 includes a biasing member 74 operably attached to door 52 to bias door to the closed position. Biasing member 74 may be a spring, hydraulic, or fluid biasing/tensioning member. A biasing connector 76, such as a cord, cable, rope, wire, or other elongated member, is operably attached to biasing member 74 and has one end fixedly attached to housing 71 and an opposing end fixedly attached as illustrated, by way of example, in FIG. 4B to door 52 for biasing door 52 toward the closed position. One or more tensioner or guide pulleys may be configured for controlling tension and movement of biasing connector 76. In one aspect, the first biasing system for door 52 includes a pulley 80 around which biasing connector 76 travels during movement of door 52 from the closed position shown, for example, in FIGS. 3A, 3B and 4A to the open position shown, for example, in FIGS. 3D and 4C. Pulley 80 may be configured to decrease travel of biasing connector 76 while increasing biasing tension on door 52 as door 52 moves to from the closed to open position. Pulley 80 may be configured to snap shut door 52 to prevent field and crop debris from entering cab 12 and/or fouling operation of testing device 30. In one aspect, first biasing system for door 52 may be include an idler pulley 84 around which biasing connector 76 travels and idler pulley 84 rotates during movement of door 52 from the closed to open position.

A second biasing system for door 54 includes a biasing member 72 operably attached to door 54 to bias door to the closed position shown, for example, in FIGS. 3A, 3B and 4A. Biasing member 72 may be a spring, hydraulic, or fluid biasing/tensioning member. A biasing connector 78, such as a cord, cable, rope, wire, or other elongated member, is operably attached to biasing member 72 and has one end fixedly attached to housing 71 and an opposing end fixedly attached as illustrated, by way of example, in FIG. 4C to door 54 for biasing door 54 toward the closed position. One or more tensioner or guide pulleys may be configured for controlling tension and movement of biasing connector 78. In one aspect, the second biasing system for door 54 includes a pulley 82 around which biasing connector 78 travels during movement of door 54 from the closed position shown, for example, in FIGS. 3A, 3B and 4A to the open position shown, for example, in FIGS. 3D and 4C. Pulley 82 may be configured to decrease travel of biasing connector 78 while increasing biasing tension on door 54 as door 54 moves to from the closed to open position. Pulley 82 may be configured to snap shut door 54 to prevent field and testing area debris from entering cab 12 and/or fouling operation of testing device 30, carriage 32, tower rail 46, and tester 34. In one aspect, second biasing system for door 54 may be include an idler pulley 86 around which biasing connector 78 travels and idler pulley 86 rotates during movement of door 54 from the closed to open position.

In another aspect of the present invention, a single biasing system, such as one of the biasing systems shown for biasing door 52 and door 54 may be configured for biasing both doors, such as configuring the first biasing system for biasing both door 52 and door 54. In another aspect, hinges 62, 64 connected to doors 52, 54 may be configured with biasing members for biasing hinges 62, 64 to a closed position thereby biasing door 52, 54 to the closed position shown, for example, in FIGS. 3A, 3B and 4A. For example, hinges 62, 64 may be a spring or tensioner hinge for biasing doors 52, 54 to the closed position. In another aspect, one or more hydraulic cylinders (not shown) may be operably attached to doors 52, 54 for hydraulically actuating doors 52, 54 from the closed position to the open position and from the open position back to the closed position using hydraulic connections 100 connected to hydraulic pump 93. In another aspect, one or more fluid or hydraulic biasing members may be operably attached to doors 52, 54 for biasing doors 52, 54 to the closed position. In another aspect, one or more coil springs may be operably attached to doors 52, 54 for biasing doors 52, 54 to the closed position.

FIGS. 6A-6D illustrate various aspects of a testing device motor enclosure 90. In one aspect, implement 10 includes a bed 14. Testing device motor enclosure 90 is removably stowed in bed 14 of implement 10. A testing device motor 92, such as a gas or electric operated motor, is mounted within testing device motor enclosure 90. A hydraulic pump 93 is mounted within testing device motor enclosure 90 and operated by testing device motor 92. In one aspect, motor 92 and hydraulic pump 93 are operably attached to a motor carriage 94. One or more bed carriage mounts 95 are attached to testing device motor enclosure 90. One or more carriage slide rails 96 are attached to bed carriage mounts 95 and motor carriage 94. Carriage slide rails 96 have a stowed position (FIG. 6A) and an unstowed position (FIG. 6B). In the stowed position, testing device motor 92 and hydraulic pump 93 are positioned within testing device motor enclosure 90 as shown, for example, in FIG. 6A. In the unstowed position, testing device motor 92 and hydraulic pump 93 are positioned generally outside of testing motor enclosure 90 as shown, for example, in FIG. 6B. Carriage slide rails 96 may be configured with a carriage slide rail release 97 for permitting movement of carriage slide rails 96 along with testing device motor 92 and hydraulic pump 93 from the stowed to unstowed position. The unstowed position allows for quick and easy access to testing device motor 92 and hydraulic pump 93 for troubleshooting, servicing, and repairing testing device motor 92 and hydraulic pump 93. One or more electrical connections 98 are operably connected to electrical connections 40 for testing device motor 92 and testing device 30. One or more electrical quick coupler connections 99 in the one or more electrical connections 98 may be configured to quickly and easily allow for testing device motor 92 to be disconnected or electrically decoupled from electrical connections 40 for testing device 30 in cab 12. One or more hydraulic connections 100 are operably connected to hydraulic pump 93 and hydraulic connections 42 for testing device 30 in cab 12. One or more hydraulic quick coupler connections 101 in the one or more hydraulic connections 100 may be configured to quickly and easily allow for hydraulic pump 93 to be disconnected or hydraulically decoupled from testing device 30 in cab 12. In one aspect, electrical quick coupler connections 99 and hydraulic quick coupler connections 101 may be disposed in a wall of testing device motor enclosure 90 shown, for example, as shown in FIG. 6C. Testing device motor enclosure 90 includes a hood 102. In one aspect, hood 102 is attached to testing device motor enclosure with one or more hinges (not shown). Hood 102 has a closed position (FIG. 6D) and an open position (FIGS. 6A and 6C). One or more hydraulic cylinders 116 attached between hood 102 and testing device motor enclosure 90 may be configured to bias hood toward the open position (FIGS. 6A and 6C). A hood latch 106 shown, for example, in FIGS. 6A and 6D may be configured within hood 102 for locking and unlocking hood 102 from testing device motor enclosure 90 to permit movement of hood between the closed and open positions. In the closed position, hood 102 prevents dust and debris from the testing area from entering testing device motor enclosure 90. Such debris presents unnecessary wear and tear on testing device motor 92 and hydraulic pump 93 and presents a fire danger when it lands on hot surfaces of testing device motor 92, such as a hot exhaust manifold. An exhaust port 112 shown, for example, in FIGS. 6A and 6D is configured in hood 102. When testing device motor 92 and hydraulic pump 93 are in the stowed position (FIG. 6A) and hood 102 is in the closed position (FIG. 6D), exhaust port 112 in hood 102 mates with the discharge orifice of exhaust manifold of testing device motor 92 to vent exhaust outside testing device motor enclosure 90. A vent 114 shown, for example, in FIGS. 6A and 6C, is configured in testing device motor enclosure 90. A fan 118 may be configured in testing device motor enclosure 90 for circulating ambient (fresh) air through enclosure 90. In one aspect, ambient air is pulled by fan 118 through vent 114 and into enclosure 90 and air within enclosure 90 is discharged from enclosure 90 by fan 118 through vent 120 disposed in a wall of testing device enclosure 90. When testing device motor 92 and hydraulic pump 93 are in the stowed position (FIG. 6A) and hood 102 is in the closed position (FIG. 6D), ambient air enters and heat exits enclosure 90 through vent 114. An air filter (not shown) may be installed at vent 114 to filter air entering enclosure 90. One or more forklift tine guides 104 shown, for example, in FIG. 6D are disposed on a bottom surface of enclosure 90 for a forklift or other similarly configured implement may remove testing device motor enclosure 90 from bed 14 of implement 10 to permit bed 14 of implement 10 to be used for other activities, tasks, or jobs. Before lifting testing device motor enclosure 90 from bed 14, electrical quick coupler connections 99 and hydraulic quick coupler connections 101 are decoupled to disconnect electrical connections 98 of testing device motor 92 and hydraulic connections 100 of hydraulic pump 93 from electrical connections 40 and hydraulic connections 42 of testing device 30 in cab 12 of implement 10. Quick coupler connections 99, 101 are recoupled for reconnecting electrical connections 40, 98 and hydraulic connections 42, 100 when testing device motor enclosure 90 is set back in bed 14 of implement 10.

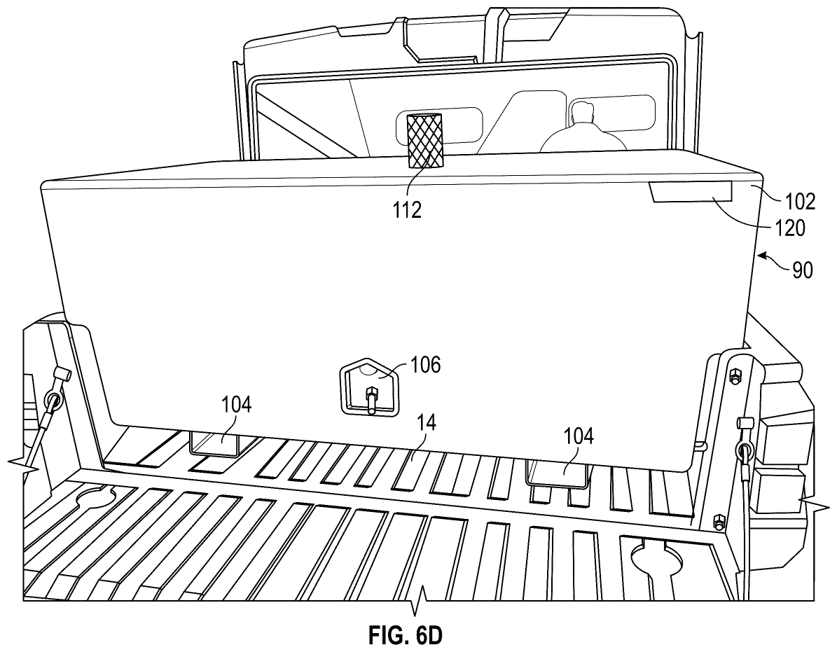

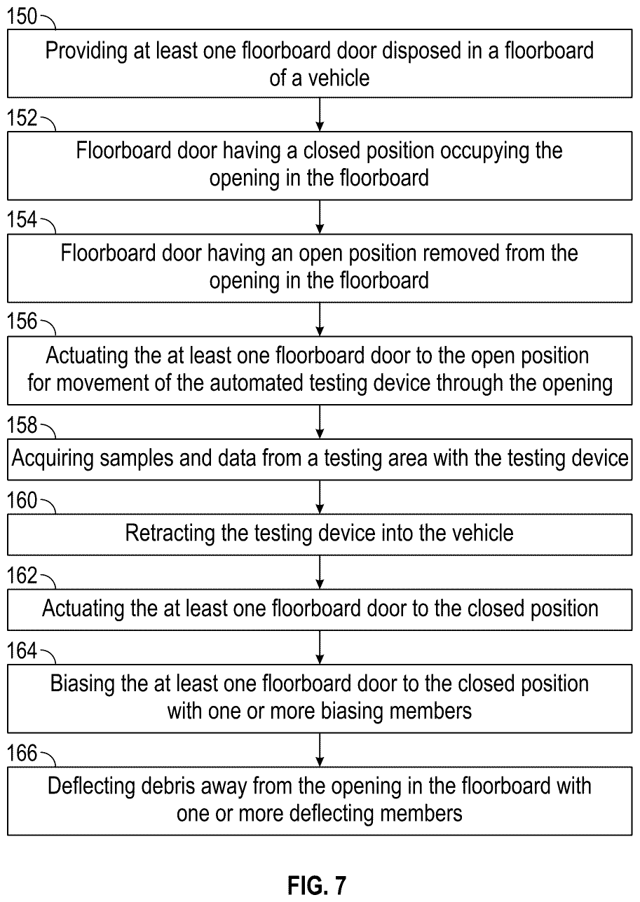

FIG. 7 illustrates operational aspects incorporating all the disclosure of FIGS. 1-6D above. Testing device 30 is configured within cab 12 for acquiring samples and/or data from outside cab 12 from the testing area below. In at least one application, samples and/or data from the testing area are collected by tester 34 as carriage 32 and tower rail 46 are lowered through opening 20 in floorboard 18. At least one floorboard door is disposed in floorboard 18 of cab 12 of an implement 10 (Step 150). Automated floorboard door 50 has a closed position occupying opening 20 in floorboard 18 (Step 152). Automated floorboard door 50 has an open position removed from opening 20 in the floorboard 18 (Step 154). For example, automated floorboard door 50 opens for data sampling with tester 34 and closes after each data sampling, where sampling may include the acquisition of a sample with a probe and/or data with a sensor, or other information and data from the testing area below. Automated floorboard door 50 is actuated to the open position for movement of the tester 34 through opening 20 (Step 156). In at least one aspect, a component of testing device 30 actuates doors 52, 54 open for tester 34 to acquire a sample and/or data from the testing area below (Step 158) and a component of door biasing system 70 actuates doors 52, 54 to a closed position (Steps 162, 164) when tester 34 retracts into cab 12 of implement 10 (Step 160). In another application, tester 34 remains within cab 12, doors 52, 54 open, tester 34 collects data and/or samples from the testing area below, and doors 52, 54 close to seal opening 20 in floorboard 18. In at least one application, collected samples from the testing area are ejected by ejector 36 into sample box 38 for collection as shown in FIG. 3B. In one aspect, port 56 in door 52 is covered by brush 58 and tester 34 passes through port 56 and brush 58 during the descending movement of carriage 32 and tower rail 46 toward opening 20 in floorboard 18. Brush 58 aids in sealing port 56 to keep dust and debris from the testing area from entering cab 12 through port 56 and aids in removing unwanted debris from being collected on tester 34 and deposited in cab 12.

In at least one exemplary operation and configuration, door 52 is actuated open as tower rail 46 descends through opening 20 toward the testing area below for tester 34 to collect a sample and/or acquire data. Tower rail 46 may be configured to actuate door 52 from the closed to open position by acting on door 52. In one aspect, descending tower rail 46 acts on cam member 60 to move door 52 to a completely open position, such as an opening angle of between 70-110 degrees relative to the position of the closed door shown, for example, in FIG. 4C. The height of cam member 60 can be increased to increase the opening angle of door 52 when actuated by the descent of tower rail 46 through opening 20 toward the testing area below. A larger opening angle of door 52 increases the separation distance between door 52 and tester 34 shown, for example, in FIG. 4C. Limiting the opening angle reduces the potential for dust and debris from the testing area below from entering cab 12 through opening 20 in floorboard 18. Limiting the opening angle of door 54 also may reduce the chances of dust and debris from the testing area below from entering cab 12 through opening 20 and fouling operation of testing device 30. As door 52 moves from the closed position (FIGS. 3A and 4A) to the open position (FIGS. 3D and 4C), biasing connector 76 is drawn downward following the opening pathway of door 52. FIG. 5A shows the position of biasing member 74 when door 52 is in the closed position (FIG. 4A) before tower rail 46 engages cam member 60 (FIG. 3B), FIG. 5B shows the position of biasing member 74 when door 52 is partially opened (FIG. 4B) by tower rail 46 (FIG. 3C), and FIG. 5C shows the position of biasing member 74 when door is completely opened (FIG. 4C) by tower rail 46 (FIG. 3D). Biasing connector 76 and door 52 retract as door 52 moves from the open position back to the closed position under the biasing action of biasing member 74. FIG. 5C shows the position of biasing member 74 when door is completely opened (FIG. 4C) by tower rail 46 (FIG. 3D) and immediately before retraction of tower rail 46, FIG. 5B shows the position of biasing member 74 when door 52 is partially closed (FIG. 4B) by retraction of tower rail 46 (FIG. 3C), and FIG. 5A shows the position of biasing member 74 when door 52 is in the closed position (FIG. 4A), tower rail 46 is retracted and no longer engages cam member 60 (FIG. 3B).

Automated floorboard door 50 may be actuated by carriage 32 passing through opening 20 in floorboard 18. In one aspect, cam member 61 on carriage 32 acts on door 54 to actuate door 54 to the open position, such as an opening angle ranging, for example, between 70-110 degrees relative to the position of door 54 when in the closed position as shown, for example, in FIG. 4C. The height of cam member 61 can be increased to increase the opening angle of door 54 when actuated by the descent of carriage 32 through opening 20 toward the testing area below. A larger opening angle of door 54 increases the separation distance between door 54 and tester 34 shown, for example, in FIG. 4C. Limiting the opening angle reduces the potential for dust and debris from entering cab 12 through opening 20 in floorboard 18. Limiting the opening angle of door 54 also reduces the chances of dust and debris from entering cab 12 through opening 20 and fouling operation of testing device 30. As door 54 moves from the closed position (FIGS. 3A-3B and 4A) to the open position (FIGS. 3D and 4C), biasing connector 78 is drawn downward following the opening pathway of door 54. FIG. 5A shows the position of biasing member 72 when door 54 is in the closed position (FIG. 4A) before cam member 61 on carriage 32 engages door 54 (FIG. 3B) and FIG. 5C shows the position of biasing member 72 when door 54 is completely opened (FIG. 4C) by cam member 61 on carriage 32 (FIG. 3C) actuating door 54 (FIG. 4C). Biasing connector 78 and door 54 retract as door 54 moves from the open position back to the closed position under the biasing action of biasing member 72. FIG. 5C shows the position of biasing member 72 when door 54 is completely opened (FIG. 4C) by cam member 61 on carriage 32 (FIG. 3C) actuating door 54 (FIG. 4C) and immediately before retraction of carriage 32 and FIG. 5A shows the position of biasing member 72 when door 54 is in the closed position (FIGS. 4A-4B), carriage 32 is retracted (FIG. 3C) and cam member 61 no longer engages door 54 (FIG. 3C).

Deflectors 66, 67 on doors 52, 54 urge dust and debris away from opening 20 in floorboard when doors 52, 54 are in the closed and open positions, and when doors 52, 54 move from the closed to open position (Step 166). In one aspect, deflectors 66, 67 are parallel to the opening edge (i.e., the edge opposite the closing edge attached to hinges 62, 64) of doors 52, 54, which aids in pushing debris away from opening 20 in floorboard 18 when doors 52, 54 are opened in a motion akin to clam shell doors opening. Keeping dust and debris from entering opening 20 prevents fouling of the operation of doors 52, 54 and testing device 30, carriage 32, tower rail 46, and tester 34. When doors 52, 54 are opened, deflectors 66, 67 aid in clearing debris away from and out of the travel path of tester 34, tower rail 46, and carriage 32. Deflector 68, positioned on the underside of floorboard 18 proximate the leading edge of opening 20 also helps deflect dust and debris coming into the path of the opening 20 by travel of implement 10 and from entering opening 20 during movement of doors 52, 54 from the closed position to the open position (Step 166).

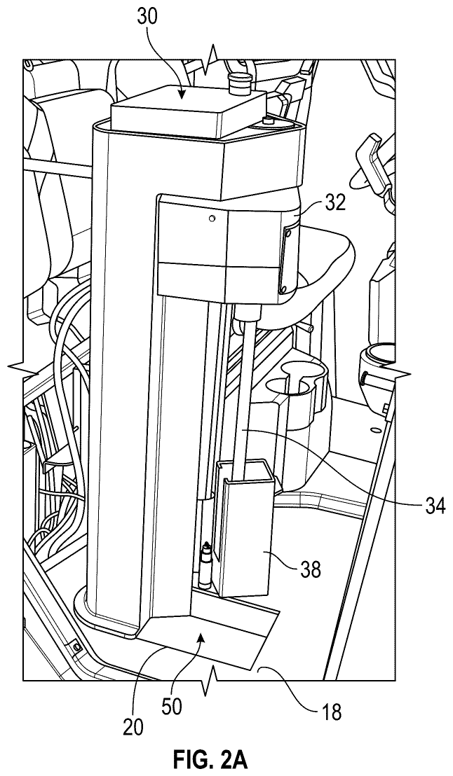

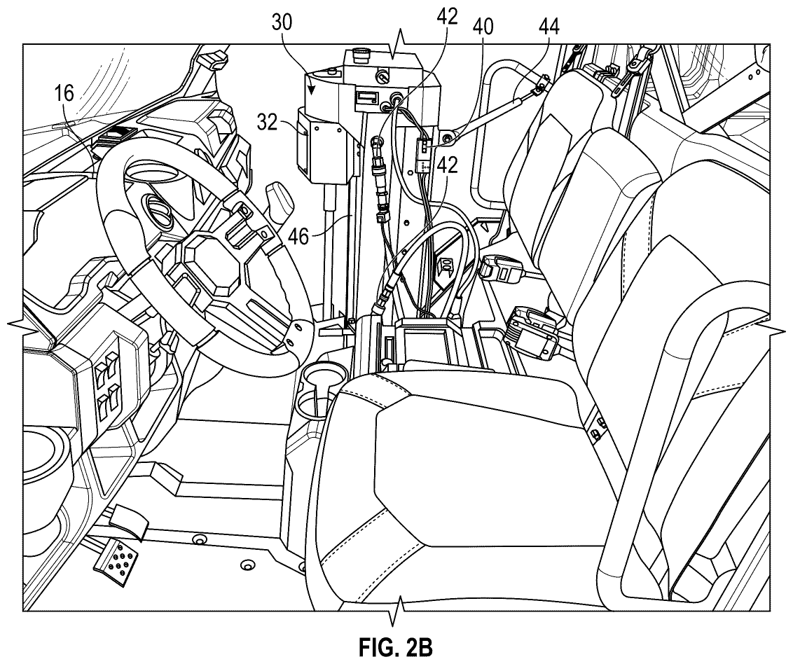

In at least one application, carriage 32 may be configured to rotate tester 34 and tower rail 46 lowers and raises carriage 32 between a testing position proximate the testing area as shown, for example, in FIG. 3D and a retracted position shown inside cab 12 as shown, for example, in FIG. 2A under hydraulic fluid pressure supplied via hydraulic connections 42 attached to testing device 30 and hydraulic connections quick coupled to hydraulic pump 93 operated by testing device motor. Testing device 30 electrical connections 40 are operably connected to operator's controls 16 and electrical connections 98 of testing device motor 92 for powering and controlling testing device 30. During sampling and/or sending, testing device motor 92 and hydraulic pump 93 are enclosed within testing device motor enclosure 90 to prevent dust and debris from the testing area from creating additional wear and tear on motor 92 and pump 93. Additionally, debris is kept from landing on hot surfaces on motor 92, such as the exhaust manifold, igniting and inadvertently starting a fire in or around the testing area. Testing device motor enclosure 90 also helps reduce operational noise in cab 12 originating from motor 92 and pump 93. Motor 92 and pump 93 may be inspected, maintained and serviced easily, quickly, and regularly by opening hood 102, releasing carriage slide rail release 97, and sliding testing device motor carriage 94 rearward toward the back and outside of bed 14. Quick coupler connections 99, 101 for electrical connections 98 and hydraulic connections 100 may be decoupled and testing device motor enclosure 90 removed from bed 14 of implement 10 for other uses of bed 14. In one aspect, a forklift, frontend loader, skid steer, or other similarly configured implement may be used to secure tine guides 104 and remove enclosure 90 from bed or alternatively place enclosure 90 within bed 14.

Testing device 30 may be configured to acquire data from the testing area below with tester 34. In one application, tester 34 readings may be taken through opening 20 in floorboard 18 when doors 52, 54 are open, by keeping tester 34, carriage 32, and tower rail 46 inside cab 12 or by moving tester 34 through opening 20 in closer proximity to the testing area below.

The invention is not to be limited to the particular embodiments described herein. In particular, the invention contemplates numerous variations in automated floorboard doors for data collection. The foregoing description has been presented for purposes of illustration and description. It is not intended to be an exhaustive list or limit any of the invention to the precise forms disclosed. It is contemplated that other alternatives or exemplary aspects are considered included in the invention. The description is merely examples of embodiments, processes or methods of the invention. It is understood that any other modifications, substitutions, and/or additions can be made, which are within the intended spirit and scope of the invention.

What is claimed is:

1. An automated floorboard door for acquiring test data from an environment outside of an implement using a testing device within the implement, comprising:
at least one floorboard door disposed in an opening in a floorboard of a implement, the at least one floorboard door having a closed position occupying the opening in the floorboard of the implement and an open position with the at least one floorboard door removed from the opening in the floorboard, wherein the at least one floorboard door is actuated to the open position for acquiring data with the testing device through the opening.

2. The automated floorboard door of claim 1, wherein the at least one floorboard door comprises a pair of floorboard doors actuated into an exterior space outside the implement.

3. The automated floorboard door of claim 1, wherein the pair of floorboard doors comprise opposing clam shell doors.

4. The automated floorboard door of claim 1, wherein the at least one floorboard door is actuated to the open position into an interior of the implement.

5. The automated floorboard door of claim 1, further comprising:
at least one biasing member operatively attached to the at least one floorboard door, wherein the at least one floorboard door is biased toward the closed position by the at least one biasing member.

6. The automated floorboard door of claim 1, wherein the at least one floorboard door is actuated to the open position in part by the testing device for acquiring data outside of the implement.

7. The automated floorboard door of claim 1, further comprising one or more debris deflectors disposed on an underside and proximate the opening in the floorboard of the implement, wherein the one or more debris deflectors deflect outside debris from entering the implement through the opening.

8. A soil probe implement, comprising:
a motorized implement having a cab with operator controls;
a soil probe operably mounted within the cab;
an opening in a floorboard of the implement for the soil probe to pass through in probing contact with the soil;
at least one floorboard door disposed in the opening in the floorboard of the implement;
wherein the at least one floorboard door is actuated to an open position for the soil probe to pass through to the soil below and a closed position for closing the opening in the floorboard when the soil probe is retracted back into the cab.

9. The soil probe implement of claim 8, wherein the at least one floorboard door comprises a pair of floorboard doors actuated toward the soil.

10. The soil probe implement of claim 8, wherein the at least one floorboard door comprises opposing clam shell doors.

11. The soil probe implement of claim 8, at least one biasing member operatively attached to the at least one floorboard door, wherein the at least one floorboard door is biased toward the closed position by the at least one biasing member.

12. The soil probe implement of claim 8, wherein the at least one floorboard door is actuated to the open position in part by the soil probe.

13. The soil probe implement of claim 8, further comprising one or more debris deflectors disposed on an underside of the motorized implement and proximate the opening in the floorboard, wherein the one or more debris deflectors deflect field debris from entering the cab through the opening.

14. The soil probe implement of claim 8, wherein the soil probe is actuated at least in part by the operator controls.

15. The soil probe implement of claim 8, further comprising:
a soil probe motor enclosure separate from the cab and motorized implement engine enclosure for housing a soil probe motor for actuating the soil probe, wherein the soil probe motor is operatively attached to one or more slide rails for sliding the soil probe motor in and out of the soil probe motor enclosure.

16. The soil probe implement of claim 15, wherein the soil probe motor enclosure includes one or more quick-connect for connecting soil probe actuation lines between the soil probe motor and the soil probe.

17. An automated method for acquiring test data from an environment outside of an implement using a testing device within the implement, comprising:
providing at least one floorboard door disposed in an opening in a floorboard of the implement, the at least one floorboard door having a closed position occupying the opening in the floorboard of the implement and an open position with the least one floorboard door removed from the opening in the floorboard;
actuating the at least one floorboard door to the open position for movement of a testing device through the opening;
acquiring data from outside of the implement with the testing device;
retracting the testing device into the implement; and
actuating the at least one floorboard door to the closed position.

18. The method of claim 17, further comprising:
biasing the at least one floorboard door to the closed position with one or more biasing members.

19. The method of claim 17, further comprising:
deflecting debris away from the opening in the floorboard with one or more deflecting members disposed on an underside and proximate the opening in the floorboard of the implement.

20. The method of claim 17, further comprising:
actuating the testing device toward the at least one floorboard door for actuating the at least one floorboard door to the open position for acquiring data from the environment outside of the implement through the opening.
